(19) United States
(12) Patent Application Publication
(10) Pub. No.: US 2016/0264954 A1
(43) Pub. Date: Sep. 15, 2016

(54) METHOD OF STABILIZING THROMBIN AND COMPOSITION THEREOF

(71) Applicant: Vitrum Biologies Inc., Road Town (VG)

(72) Inventors: Cheng-Yao SU, Taipei City (TW); Chung Chin SUN, Taipei City (TW)

(73) Assignee: Vitrum Biologies Inc., Road Town (VG)

(21) Appl. No.: 15/069,177

(22) Filed: Mar. 14, 2016

Related U.S. Application Data
(60) Provisional application No. 62/132,751, filed on Mar. 13, 2015.

(51) Int. Cl. C12N 9/74

(52) U.S. Cl. CPC C12N 9/6429; C12Y 304/21005

(57) ABSTRACT

A method of stabilizing thrombin in a thrombin solution is provided. The method comprises heating the thrombin solution at a temperature of 35-85° C. for 1 to 20 seconds, and quenching the heating. Also provided is a stabilized thrombin composition. The stabilized thrombin composition is characterized in that it is prepared by heating a thrombin solution at a temperature of 35-85° C. for 1 to 20 seconds, and quenching the heating. The thrombin solution may be further lyophilized to obtain a stabilized thrombin composition in lyophilized form.

### FIELD OF THE INVENTION

The present invention pertains to a method of stabilizing thrombin in a thrombin solution.

### BACKGROUND OF THE INVENTION

Thrombin is a serine protease which plays a key role in the process of blood clotting. Prothrombin is cleaved to form thrombin in the coagulation cascade, which ultimately results in the reduction of blood loss. Thrombin in turn acts as a serine protease to convert soluble fibrinogen into insoluble fibrin which promotes blood clotting.

However, thrombin is unstable in nature and its activity for blood clotting drops significantly even after only 2-3 hours after freshly prepared. There have been extensive attempts to stabilize thrombin but they are substantially chemical ones. For example, EP 1221479 B1 disclosing the use of a nonionic surfactant and calcium ions, and U.S. Pat. No. 8,071,090 B2 disclosing utilizing a preservative of benzyl alcohol or chlorobutanol, and sucrose.

In view of that the chemicals used to stabilize thrombin is usually not biocompatible, there is still a need for a safer and more biocompatible method to prepare a thrombin solution or composition for clinical use.

### BRIEF SUMMARY OF THE INVENTION

It is unexpectedly found in the present invention that thrombin in a thrombin solution can be stabilized through a physical treatment, that is, a brief heating treatment.

Accordingly, in one aspect, the present invention features a method of stabilizing thrombin in a thrombin solution, comprising heating the thrombin solution at a temperature of 35-85° C. for 1 to 20 seconds, and quenching the heating.

In another aspect, the present invention provides a stabilized thrombin composition prepared by a method of the present invention.

In certain embodiments of the present invention, the method further comprises a step of quenching the heating.

According to the present invention, a stabilized thrombin solution or composition may be further lyophilized to take a lyophilized from.

It is to be understood that both the foregoing general description and the following detailed description are exemplary and explanatory only and are not restrictive of the invention.

### DETAILED DESCRIPTION OF THE INVENTION

The present invention is based on the unexpected finding that thrombin can be stabilized by a heating treatment.

In one aspect, the present invention provides a method of stabilizing thrombin in a thrombin solution, comprising heating the thrombin solution at a temperature of 35-85° C. for 1 to 20 seconds, and quenching the heating.

The thrombin solution may be prepared from blood through a method known in the art. In one example, 6-7 ml of thrombin solution is prepared from about 10 mL of platelet poor plasma (PPP) by a thrombin generation device.

According to one embodiment, the heating is performed in a water bath. For example, 1.5 ml of thrombin solution in an Eppendorf may be placed in a 35-85° C. water bath for 1 to 20 seconds and then quenched the heating in iced water to obtain a stabilized thrombin solution.

Appropriate combination of heating temperature and time may be determined by a person of ordinary skill in the art. For example, a higher heating temperature in combination with a shorter heating time, and a lower heating temperature in combination with a longer heating time.

According to the present invention, the step of quenching is to stop the heating treatment by bring the temperature of the thrombin solution back to that before the heating treatment, i.e. to room temperature or a lower one, for example, a temperature of 0-25° C. The quenching step may be performed by bathing the thrombin solution in an iced solution, for example, iced water.

In general, untreated thrombin solution loses its clotting activity after about 2 hours after freshly prepared. However, a thrombin solution stabilized by the method of the present invention maintains its clotting activity after 2 hours after freshly prepared, and maintains its clotting activity even when it is reconstituted from a lyophilized form thereof.

In another aspect, the present invention provides a stabilized thrombin composition prepared by heating a thrombin solution at a temperature of 35-85° C. for 1 to 20 seconds, and quenching the heating, to obtain the stabilized thrombin composition.

In certain preferred embodiments, the thrombin solution is further lyophilized to obtain a stabilized thrombin composition in lyophilized form.

Accordingly, the present invention also provides a lyophilized stabilized thrombin composition, which is prepared through a process comprising the following steps: (i) heating a thrombin solution at a temperature of 35-85° C. for 1 to 20 seconds, and quenching the heating, to obtain a stabilized thrombin solution; and (ii) subjecting the stabilized thrombin solution to lyophilization.

The present invention is further illustrated by the following examples, which are provided for the purpose of demonstration rather than limitation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method of stabilizing thrombin in a thrombin solution, comprising heating the thrombin solution at a temperature of 35-85° C. for 1 to 20 seconds, and quenching the heating.

2. The method of claim 1, wherein the quenching is performed by bathing the thrombin solution in an iced solution.

3. A stabilized thrombin composition prepared by heating a thrombin solution at a temperature of 35-85° C. for 1 to 20 seconds, and quenching the heating.

4. The stabilized thrombin composition of claim 3 in lyophilized form.
